SEQUENCE LISTING (21 sequences)

SEQ ID NO: 1 - DNA, 618 bp, Homo sapiens
atggaaaacgaattgttctgctcgggcgtcctggtgcatccgcagtgggtgctgtcagccgcacactgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggcagaatgcctaccgtgctgcagtgcgtgaacgtgtcggtggtgtctgaggaggtctgcagtaagctctatgacccgctgtaccaccccagcatgttctgcgccggcggagggcaagaccagaaggactcctgcaacggtgactctggggggcccctgatctgcaacgggtacttgcagggccttgtgtctttcggaaaagccccgtgtggccaagttggcgtgccaggtgtctacaccaacctctgcaaattcactgagtggatagagaaaaccgtccaggccagttaa

SEQ ID NO: 2 - DNA, 481 bp, Homo sapiens
atggaaaacgaattgttctgctcgggcgtcctggtgcatccgcagtgggtgctgtcagccgcacactgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacgggtgactctggggggcccctgatctgcaacgggtacttgcagggccttgtgtctttcggaaaagccccgtgtggccaagttggcgtgccaggtgtctacaccaacctctgcaaattcactgagtggatagagaaaaccgtccaggccagttaa

SEQ ID NO: 3 - DNA, 702 bp, Homo sapiens
atggaaaacgaattgttctgctcgggcgtcctggtgcatccgcagtgggtgctgtcagccgcacactgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggtgagctcacgggtgtgtgtctgccctcttcaaggaggtcctctgcccagtcgcgggggctgacccagagctctgcgtcccaggcagaatgcctaccgtgctgcagtgcgtgaacgtgtcggtggtgtctgaggaggtctgcagtaagctctatgacccgctgtaccaccccagcatgttctgcgccggcggagggcaagaccagaaggactcctgcaacggtgactctgggggggccctgatctgcaacgggtacttgcagggccttgtgtctttcggaaaagccccgtgttggccaagttggcgtgccaggtgtctacaccaacctctgcaaattcactgagtggatagagaaaaccgtccaggccagttaa

SEQ ID NO: 4 - DNA, 834 bp, Homo sapiens
ggaatgagcctggatccggggagcccagagggaagggctgggaggcgggaatcttgcttcggaaggactcagagagtcctgacttgaaatctcagcccagtgctgagtctctagtgaactaagctcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggtgaactcacgggtgtgtgtctgccctcttcaaggaggtcctctgcccagtcgcgggggctgacccagagctctgcgtcccaggcagaatgcctaccgtgctgcagtgcgtgaacgtgtcggtggtgtctgaggaggtctgcagtaagctctatgacccgctgtaccaccccagcatgttctgcgccggcggagggcaagaccagaaggactcctgcaacggtgactctggggggcccctgatctgcaacgggtacttgcagggccttgtgtctttcggaaaagccccgtgtggccaagttggcgtgccaggtgtctacaccaacctctgcaaattcactgagtggatagagaaaaccgtccaggccagttaactctggggactgggaacccatgaaattgacccccaaatacatcctgcggaaggaattcaggaatatctgatcccagcccctcctccc

SEQ ID NO: 5 - DNA, 440 bp, Homo sapiens
ggaatgagcctggatccggggagcccagagggaagggctgggaggcgggaatcttgcttcggaaggactcagagagccctgacttgaaatctcagcccagtgctgagtctctagtgaactaagctcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggcagaatgcctaccgtgctgcagtgcgtgaacgtgtcggtggtgtctgaggaggtctgcagtaagc

SEQ ID NO: 6 - DNA, 457 bp, Homo sapiens
ggctctgggaggaggacggaatgagcctggatccggggagcccagagggaagggctgggaggcgggaatcttgcttcggaaggactcagagagccctgacttgaaatctcagcccagtgctgagtctctagtgaactaagctcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggcagaatgcctaccgtgctgcagtgcgtgaacgtgtcggtggtgtctgaggaggtctgcagtaagc

SEQ ID NO: 7 - DNA, 636 bp, Homo sapiens
accaccccagcatgttctgcgccggcggagagcaagaccagaaggactcctgcaacgtgagagaggggaaaggggagggcaggcgactcagggaagggtggagaagggggagacagagacacacagggccgcatggcgagatgcagagatggagagacacacagggagacagtgacaactagagagagaaactgagagaaacagggaaataaacacaggaataaagagaagcaaaggaagagagaaacagaaacagacatgggggaggcagaaacacacacacatagaaatgcagctgaccttccaacagcatggggcctgagggcggtgacctccacccaacagaaaatcctcttataacttttgactccccaaaaaacctgactagaaatagcctactgttgacgggggagccttaccaataacataaatagtcgatttatgcatacgttttatgcattcatgatatacctttgttggaattttttgatatttctaagctacacagttcgtctgtgaatttttttaaattgttgcaactctcctaaaattttttctaatgtgtttattgaaaaaaatccaagtataagtggacttgtgcagttcaaaccagggttgttcaagggtcaactgtgt

SEQ ID NO: 8 - protein, 205 aa, Homo sapiens
MENELFCSGVLVHPQWVLSAAHCFQNSYTIGLGLHSLEADQEPGSQMVEASLSVRHPEYNRPLLANDLMLIKLDESVSESDTIRSISIASQCPTAGNSCLVSGWGLLANGRMPTVLQCVNVSVVSEEVCSKLYDPLYHPSMFCAGGGQDQKDSCNGDSGGPLICNGYLQGLVSFGKAPCGQVGVPGVYTNLCKFTEWIEKTVQAS

SEQ ID NO: 9 - protein, 110 aa, Homo sapiens
MENELFCSGVLVHPQWVLSAAHCFQNSYTIGLGLHSLEADQEPGSQMVEASLSVRHPEYNRPLLANDLMLIKLDESVSESDTIRSISIASQCPTAGNSCLVSGWGLLANG

SEQ ID NO: 10 - protein, 146 aa, Homo sapiens
MENELFCSGVLVHPQWVLSAAHCFQNSYTIGLGLHSLEADQEPGSQMVEASLSVRHPEYNRPLLANDLMLIKLDESVSESDTIRSISIASQCPTAGNSCLVSGWGLLANGELTGVCLPSSRRSSAQSRGLTQSSASQAECLPCCSA

SEQ ID NO: 11 - protein, 100 aa, Homo sapiens [sequence omitted]

SEQ ID NO: 12 - protein, 85 aa, Homo sapiens
MVEASLSVRHPEYNRPLLANDLMLIKLDESVSESDTIRSISIASQCPTAGNSCLVSGWGLLANGRMPTVLQCVNVSVVSEEVCSK

SEQ ID NO: 13 - protein, 85 aa, Homo sapiens; identical to SEQ ID NO: 12

SEQ ID NO: 14 - protein, 129 aa, Homo sapiens
AISSQVFWGVKSYKRIFCWVEVTALRPHAVGRSAAFLCVCVSDMSVSVSLFLCFSLFLCLFPCFSQFLSLVVTVSLCVSPSLHLAMRPCVSLSPPSPPFPESPALPFPLSHVAGVLLVLLSAGAEHAGV

SEQ ID NO: 15 - RNA, 618 nt, Homo sapiens
auggaaaacgaauuguucugcucgggcguccuggugcauccgcagugggugcugucagccgcacacuguuuccagaacuccuacaccaucgggcugggccugcacagucuugaggccgaccaagagccagggagccagaugguggaggccagccucuccguacggcacccagaguacaacagacccuugcucgcuaacgaccucaugcucaucaaguuggacgaauccguguccgagucugacaccauccggagcaucagcauugcuucgcagugcccuaccgcggggaacucuugccucguuucuggcuggggucugcuggcgaacggcagaaugccuaccgugcugcagugcgugaacgugucgguggugucugaggaggucugcaguaagcucuaugacccgcuguaccaccccagcauguucugcgccggcggagggcaagaccagaaggacuccugcaacggugacucuggggggccccugaucugcaacggguacuugcagggccuugugucuuucggaaaagccccguguggccaaguuggcgugccaggugucuacaccaaccucugcaaauucacugaguggauagagaaaaccguccaggccaguuaa

SEQ ID NO: 16 - RNA, 480 nt, Homo sapiens
auggaaaacgaauuguucugcucgggcguccuggugcauccgcagugggugcugucagccgcacacuguuuccagaacuccuacaccaucgggcugggccugcacagucuugaggccgaccaagagccagggagccagaugguggaggccagccucuccguacggcacccagaguacaacagacccuugcucgcuaacgaccucaugcucaucaaguuggacgaauccguguccgagucugacaccauccggagcaucagcauugcuucgcagugcccuaccgcggggaacucuugccucguuucuggcuggggucugcuggcgaacgggugacucuggggggccccugaucugcaacggguacuugcagggccuuggucuuucggaaaagccccguguggccaaguuggcgugccaggugucuacaccaaccucugcaaauucacugaguggauagagaaaaccguccaggccaguuaa

SEQ ID NO: 17 - RNA, 701 nt, Homo sapiens
auggaaaacgaauuguucugcucgggcguccuggugcauccgcagugggugcugucagccgcacacuguuuccagaacuccuacaccaucgggcugggccugcacagucuugaggccgaccaagagccagggagccagaugguggaggccagccucuccguacggcacccagaguacaacagacccuugcucgcuaacgaccucagcucaucaaguuggacgaauccguguccgagucugacaccauccggagcaucagcauugcuucgcagugcccuaccgcggggaacucuugccucguuucuggcuggggucugcuggcgaacggugagcucacgggugugugucugcccucuucaaggagguccucugcccagucgcgggggcugacccagagcucugcgucccaggcagaaugccuaccgugcugcagugcgugaacgugucgguggugucugaggaggucugcaguaagcucuaugacccgcuguaccaccccagcauguucugcgccggcggagggcaagaccagaaggacuccugcaacggugacucugggggggcccugaucugcaacggguacuugcagggccuugugucuuucggaaaagccccguguuggccaaguuggcgugccaggugucuacaccaaccucugcaaauucacugaguggauagagaaaaccguccaggccaguuaa

SEQ ID NO: 18 - RNA, 830 nt, Homo sapiens
ggaaugagccuggauccggggagcccagagggaagggcugggaggcgggaaucuugcuucggaaggacucagagaguccgacuugaaaucucagcccagugcugagucucuagugaacuaagcuccuacaccaucgggcugggccugcacagucuugaggccgaccaagagccagggagccagagguggaggccagccucuccguacggcacccagaguacaacagacccuugcucgcuaacgaccucaugcucaucaaguuggacgaauccguguccgagucugacaccauccggagcaucagcauugcuucgcagugcccuaccgcggggaacucuugccucguuucuggcuggggucugcuggcgaacgggaacucacgggugugugucugcccucuucaaggagguccucugcccagucgcgggggcugacccagagcucugcgucccaggcagaagccuaccgugcugcagugcgugaacgugucgguggugucugaggaggucugcaguaagcucuaugacccgcuguaccaccccagcauguucugcgccggcggagggcaagaccagaaggacuccugcaacggugacucuggggggccccugaucugcaacggguacuugcagggccuugugucuuucggaaaagccccguguggccaaguuggcgugccaggugucuacaccaaccucugcaaauucacugaguggauagagaaaaccguccaggccaguuaacucuggggacugggaacccaugaaauugacccccaaauacauccugcggaaggaauucaggaauaucugaucccagccccuccuccc

SEQ ID NO: 19 - RNA, 438 nt, Homo sapiens
ggaaugagccuggauccggggagcccagagggaagggcgggaggcgggaaucuugcuucggaaggacucagagagcccugacuugaaaucucagcccagugcugagucucuagugaacuaagcuccuacaccacgggcugggccugcacagucuugaggccgaccaagagccagggagccagaugguggaggccagccucuccguacggcacccagaguacaacagacccuugcucgcuaacgaccucaugcucaucaaguuggacgaauccguguccgagucugacaccauccggagcaucagcauugcuucgcagugcccuaccgcggggaacucuugccucguuucuggcuggggucugcuggcgaacggcagaaugccuaccgugcugcagugcgugaacgugucgguggugucugaggaggucugcaguaagc

SEQ ID NO: 20 - RNA, 455 nt, Homo sapiens
gcucugggaggaggacggaaugagccuggauccggggagcccagagggaagggcugggaggcgggaaucuugcuucggaaggacucagagagcccugacuugaaaucucagcccagugcugagucucuagugaacuaagcuccuacaccaucgggcugggccugcacagucuugaggccgaccaagagccagggagccagaugguggaggccagccucuccguacggcacccagaguacaacagacccuugcucgcuaacgaccucaugccaucaaguuggacgaauccguguccgagucugacaccauccggagcaucagcauugcuucgcagugcccuaccgcggggaacucuugccucguuucuggcuggggucugcuggcgaacggcagaaugccuaccgugcugcagugcgugaacgugucgguggugucugaggaggucugcaguaagc

SEQ ID NO: 21 - RNA, 635 nt, Homo sapiens
accaccccagcauguucugcgccggcggagagcaagaccagaaggacuccugcaacgugagagaggggaaaggggagggcaggcgacucagggaaggguggagaagggggagacagagacacacagggccgcauggcgagaugcagagauggagagacacacagggagacagugacaacuagagagagaaacugagagaaacagggaaauaaacacaggaauaaagagaagcaaaggaagagagaaacagaaacagacaugggggaggcagaaacacacacacauagaaaugcagcugaccuuccaacagcauggggccugagggcggugaccuccacccaacagaaaauccucuuauaacuuuugacuccccaaaaaaccugacuagaaauagccuacuguugacgggggagccuuaccaauaacauaaauagucgauuuaugcauacguuuuaugcauucaugauauaccuuuguuggaauuuuuugauauuucuaagcuacacaguucgucugugaauuuuuuuaaauuguugcaacucuccuaaaauuuuuucuaauguguuuauugaaaaaaauccaaguaaaguggacuugugcaguucaaaccaggguuguucaagggucaacugugu

(19) United States
(12) Patent Application Publication
(10) Pub. No.: US 2005/0106643 A1
(43) Pub. Date: May 19, 2005

(54) Differentially expressed genes in prostate cancer

(76) Inventor: Fahri Saatcioglu, Oslo (NO)

Correspondence Address: CLARK & ELBING LLP, 101 FEDERAL STREET, BOSTON, MA, 02110, US

(21) Appl. No.: 10/726,093

(22) Filed: Dec. 1, 2003

Related U.S. Application Data
(63) Continuation of application No. 09/743,682, filed on Jan. 10, 2001, now abandoned.
(60) Provisional application No. 60/135,325, filed on May 20, 1999.
(60) Provisional application No. 60/135,333, filed on May 20, 1999.

(51) Int. Cl. G01N 33/574

(52) U.S. Cl. 435/7.23

(57) ABSTRACT

SEQ ID NO: 1, SEQ ID NO: 2, and SEQ ID NO: 3 encode an intracellular protein that is expressed in prostate epithelial cells in a hormone dependent manner. Encoded proteins SEQ ID NO: 4, SEQ ID NO: 5, and SEQ ID NO: 6 have predominantly perinuclear, nuclear and predominantly nuclear location localization within a cell, respectively. In contemplated methods of detecting a neoplastic cell in a system, a predetermined amount of at least one of SEQ ID NO: 4, SEQ ID NO:5, and SEQ ID NO: 6, or at least one of SEQ ID NO: 7, SEQ ID NO: 8, and SEQ ID NO: 9 is correlated with the presence of a neoplastic cell and detected within the system employing specific binding of a labeled probe. In a method of identifying differentially expressed genes, a tissue specific array of cDNA prepared by suppression subtractive hybridization is arranged on a solid phase. Two nucleic acid preparations are individually hybridized with the array, wherein the first and second nucleic acid preparations are prepared from treated and untreated target tissue. A comparison of the hybridization patterns reveals differentially expressed genes.

This application claims the benefit of U.S. provisional application No. 60/135,325 filed May 20, 1999, and U.S. provisional application No. 60/135,333 filed May 20, 1999, both of which are incorporated herein by reference in their entirety.

### FIELD OF THE INVENTION

The field of the invention is neoplastic diseases, and especially detection and therapy of prostate cancer.

### BACKGROUND OF THE INVENTION

Prostate cancer has become the most commonly diagnosed malignancy in males in the western world, and is the second most common cause of cancer death among men in Europe and the United States (Boring, C. C., Squires, T. S., and Tong, T. (1993). Cancer J. Gun. 43, 7-26; Carter, 1-LB., Pianpadosi, S., and Isaacs, J. T. (1990). J. Urol. 143, 742-746). Worse yet, in recent years the annual incidence rate of newly diagnosed prostate cancer, as well as the number of prostate cancer deaths continuously rose.

Androgens not only play a key role in the development and maintenance of the normal prostate, but also in the initiation and progression of prostate cancer (Moore, R. A. (1944). Surgery 16, 152-167; Huggins, C., and Johnson, M. A. (1947). J. Am. Med. Assoc. 135, 1146-1152). For example, androgens typically induce cell proliferation and inhibit cell death in the healthy prostate gland. Withdrawal of androgens stops proliferation of cells and induces apoptosis with concomitant involution of the prostate gland. Involution upon androgen withdrawal is generally a characteristic of a normal prostate gland as well as of a prostate tumor in the early stages of the disease, when the tumor still remains androgen dependent. Consequently, androgen withdrawal treatment is commonly used to reverse tumor growth. However, in the case of many prostate tumors, the tumor recurs after a few months or years almost invariably in an androgen insensitive state. At this point, successful therapy of prostate cancer is difficult and prognosis for survival is usually relatively low.

Almost all of the biological effects of androgens are mediated by the androgen receptor, a hormone-activated transcription factor. Even though the androgen receptor was cloned over ten years ago, the mechanisms by which the androgen receptor regulates gene expression is not well-understood. Furthermore, only a very few of its target genes have been identified, including the prostate specific antigen (PSA), the related glandular kallikrein 2 (hKLK2), and the androgen receptor itself. Another androgen regulated protein, a secreted serine protease with prostate restricted expression, termed “prostase” was recently described by Nelson et al. (Nelson, P. S., et al. (1999) Proc. Natl. Acad. Sci. 96, 3114-3119). However, the biological functions of the proteins coded by the PSA, hKLK2 and prostase genes are poorly understood at present.

Adding to the difficulties in understanding the role of androgens in prostate cancer is that in vivo and in vitro model systems frequently do not closely mimic the human disease. Furthermore, close homologues of the PSA gene, the only marker for prostate cancer in human, are not known in other animal species. Moreover, in vitro studies are hampered due to the limited number of cell lines that have been derived from human prostate. For example, the only androgen sensitive and androgen responsive cell line that is widely used is LNCaP, characterized by cells originally derived from a lymph node metastasis of a human prostate carcinoma (Horoszewicz, J. S., Leong, S. S., Kawinski, E., Karr, J. P., Rosenthal, H., Chu, T. M., Mirand, E. A., and Murphy, G. P. (1983). Cancer Res. 43, 1809-1818).

In spite of numerous studies on the effects of androgens in the role of prostate cancer, relatively little is known about the molecular genetic effects of androgens in prostate cells. More detailed knowledge about androgen responsive genes and their role in signal transduction, as well as gross morphological and physiological transformations will potentially result in better diagnostic tools, and provide possible new targets for a drug-based therapy of prostate cancer. Therefore, there is still a need to provide improved methods to identify androgen responsive, differentially expressed genes in prostate cancer.

### SUMMARY OF THE INVENTION

The present invention is directed to differentially expressed genes in neoplastic cells, and particularly relates to hormone dependent genes in prostate cancer. The polynucleotides with the SEQ ID NO:1-SEQ ID NO: 7 encode an intracellular protein, and while the corresponding polypeptides SEQ ID NO:11-SEQ ID NO:14 have an intracellular location, the corresponding expression products SEQ ID NO:8-SEQ ID NO: 10 have predominantly perinuclear, nuclear and predominantly nuclear localization within a cell, respectively.

In one aspect of the inventive subject matter, SEQ ID NO:1-SEQ ID NO:7 are expressed in prostate cancer cells in a hormone dependent manner.

In another aspect of the inventive subject matter, a method of detecting a neoplastic cell in a system includes a step in which a predetermined amount of an RNA comprising at least one of SEQ ID NO:15-SEQ ID NO:21 is correlated with the presence of a neoplastic cell, and the predetermined amount, or more, is subsequently detected in the system. Contemplated detection methods preferably employ a labeled probe that is detectable via fluorescence detection, luminescence detection, scintigraphy, autoradiography, or formation of a dye. Alternative preferred detection methods include addition of at least one nucleotide to the probe (e.g., PCR, LCR).

In a further aspect of the inventive subject matter, a method of detecting a neoplastic cell in a system includes a step in which a predetermined amount of a polypeptide comprising at least one of SEQ ID NO:8-SEQ ID NO:14 is correlated with the presence of a neoplastic cell, and the predetermined amount, or more, is subsequently detected in the system. Contemplated detection methods preferably employ a labeled probe that is detectable via fluorescence detection, luminescence detection, scintigraphy, autoradiography, or formation of a dye, and preferred probes include antibodies, antibody fragments, and natural and synthetic ligands of the polypeptide.

In a still further aspect of the inventive subject matter, a method of identifying differentially expressed genes in a target tissue has one step in which a target tissue-specific cDNA library is prepared by suppression subtractive hybridization, and a plurality of genes from the library is immobilized on a solid phase. Nucleic acid preparations from treated and untreated target tissue are individually hybridized with array, respectively, and hybridization patterns are compared to identify differentially expressed genes.

Various objects, features, aspects and advantages of the present invention will become more apparent from the following detailed description of preferred embodiments of the invention, along with the accompanying drawing.

### BRIEF DESCRIPTION OF THE DRAWING

FIGS. 1A and 1B depict schematic nucleic acid and amino acid based multiple sequence alignments of SEQ ID NO:1-SEQ ID NO:7 and SEQ ID NO:8-SEQ ID NO:13, respectively.

FIG. 2 is a photograph of an exemplary reverse northern blot of several clones from a cDNA library of androgen treated prostate cancer cells.

FIG. 3 is a photograph of an exemplary multiple tissue northern blot of one of the isolated polynucleotides.

FIG. 4 is a photograph of an agarose gel after electrophoretic separation of splicing variants of SEQ ID NO:1.

FIG. 5 is a series of photomicrographs illustrating the intracellular localization of GFP-fusion proteins of polypeptides of SEQ ID NO:8-SEQ ID NO:10.

FIG. 6 is an autoradiograph of a northern blot indicating hormone dependent expression of SEQ ID NO:1-SEQ ID NO:7.

### DETAILED DESCRIPTION

As used herein, the term “intracellular protein” refers to a protein that is expressed and retained within a cell irrespective of its subcellular localization. For example, DNA polymerase (nucleus), glyceraldehyde-3-phosphate dehydrogenase (cytoplasm), and cytochrome c oxidase (mitochondria) are all considered intracellular proteins. In contrast, the term “extracellular protein” refers to a protein that is exported from a cell. There are various mechanisms by which a protein can be exported from a cell, all of which are contemplated herein. For example, while many exported proteins have a signal sequence that allows specific export of the protein across a cell membrane, other proteins are exported through vesicles via the endoplasmatic reticulum, etc.

As also used herein, a protein has a “predominantly perinuclear localization” when a majority of the protein (i.e., more than 50% of the total amount as fluorimetrically detectable by GFP-fusion) is located in a volume around the nucleus that does not exceed a volume greater than three volumes of the nucleus. A protein has a “predominantly nuclear localization” when a majority of the protein is located within the nucleus. In contrast, the term “nuclear localization” means that substantially all of the detectable protein is located within the nucleus. Localization of a protein can be determined fluorimetrically by GFP-fusion (GFP: green fluorescence protein).

Employing a novel combined SSH-reverse northern blot procedure (first disclosed in provisional application 60/135,325 filed May 20, 1999), we isolated seven cDNAs (SEQ ID NO:1-SEQ ID NO:7) from human prostate cancer cells. All seven cDNA molecules code for an intracellular protein, which is expressed in an androgen and glucocorticoid dependent manner. Remarkably, the sequences SEQ ID NO:1-SEQ ID NO:7 show a high degree of homology/identity with prostase, a previously reported serine protease (Nelson, P. S., et al. (1999) Proc. Natl. Acad. Sci. 96, 3114-3119). However, in comparison with the prostase, all of the sequences SEQ ID NO:1-SEQ ID NO:7 lack a 49 amino acid N-terminal portion corresponding to the first exon of the prostase. It should be especially appreciated that the first exon of the prostase not only includes structurally important amino acids, but also includes a signal peptide sequence that renders the serine protease a secreted, extracellular enzyme. Consequently, the cDNA molecules with the sequence of SEQ ID NO:1-SEQ ID NO:7 encode intracellular proteins that are functionally and structurally different from the prostase, and are therefore independent and novel genes.

With respect to the base composition of SEQ ID NO:1-SEQ ID NO:7, nucleotide sequences other that SEQ ID NO:1-SEQ ID NO:7 are also contemplated and particularly include variations of SEQ ID NO:1-SEQ ID NO:7 that include point mutations, insertions, deletions and any reasonable combination thereof, so long as alternative sequences encode an intracellular protein. Therefore, polynucleotides are contemplated that have at least 80%, preferably at least 85%, more preferably at least 90% and most preferably at least 95% identity with the sequences of SEQ ID NO:1-SEQ ID NO:7, so long as contemplated polynucleotides encode an intracellular protein.

For example, point mutations may arise at any position of the sequence from an apurinic, apyrimidinic, or otherwise structurally impaired site within the cDNA. Alternatively, point mutations may be introduced by random or site-directed mutagenesis procedures (e.g., oligonucleotide assisted or by error prone PCR). Likewise, deletions and/or insertions may be introduced into the sequences, and particularly preferred insertions comprise 5′- and/or 3′-fusions with a polynucleotide that encodes a reporter moiety or an affinity moiety. Other particularly preferred insertions comprise a nucleic acid that further includes functional elements such as a promoter, enhancer, hormone responsive element, origin of replication, transcription and translation initiation sites, etc. It should especially be appreciated that where insertions with one or more functional elements are present, the resulting nucleic acid may be linear or circular (e.g., transcription or expression cassettes, plasmids, etc.).

Still further contemplated variations include substitution of one or more atoms or chemical groups in the sequence with a radioactive atom or group. For example, where contemplated cDNAs are employed as a hybridization-specific probes, a fluorophor or enzyme (e.g., β-galactosidase for generation of a dye, or luciferase for generation of luminescence) may be coupled to the sequence to identify position and/or quantity of a complementary sequence. Alternatively, where contemplated cDNA molecules are utilized for affinity isolation procedures, the cDNA may be coupled to a molecule that is known to have a high-affinity (i.e., K_{d}<10⁻⁴ mol⁻¹) partner, such as biotin, or an oligo-histidyl tag. In another example, one or more phosphate groups may be exchanged for a radioactive phosphate group with a ³²P or ³³P isotope to assist in detection and quantification, where the radiolabeled cDNA is employed as a hybridization probe.

It is also contemplated that the polypeptides (encoded by SEQ ID NO:1-SEQ ID NO:7) having the peptide sequence of SEQ ID NO:8-SEQ ID NO:14 may be produced in vivo or in vitro, and may be chemically and/or enzymatically modified. Contemplated polypeptides can be isolated from prostate tissue or prostate cancer cells that may or may not be in a hormone dependent state. Alternatively, and especially where larger amounts (i.e., >10 mg) are desirable, recombinant production (e.g., in a bacterial, yeast, insect cell, or mammalian cell system) may advantageously be employed to generate significant quantities of contemplated polypeptides.

It should further be appreciated that recombinant production not only offers a more economical strategy to produce contemplated polypeptides, but also allows specific modification in the amino acid sequence and composition to tailor particular biochemical, catalytic and physical properties. For example, where increased solubility of contemplated polypeptides is desirable, one or more hydrophobic amino acids may be replaced with hydrophilic amino acids. Alternatively, where reduced or increased catalytic activity is required, one or more amino acids may be replaced or eliminated. In still another example, fusion proteins with contemplated proteins are contemplated, in which an additional polypeptide is added to the N-terminus and/or C-terminus of contemplated polypeptide. Particularly contemplated fusion proteins include fusions with enzymatically active fusion partners (e.g., for dye formation or substrate conversion) and fluorescent fusion partners such as GFP, EGFB, BFP, etc. Therefore, sequences other than the sequences of SEQ ID NO:8-SEQ ID NO:14 are also contemplated, so long as the polypeptides are intracellular proteins, and alternative peptide sequences may have a sequence that has a 70%, preferably 80%, more preferably 90%, and most preferably 95% homology to the sequences of SEQ ID NO:8-SEQ ID NO:14.

With respect to chemical and enzymatic modifications of contemplated polypeptides, it is contemplated that many modifications are appropriate, including addition of mono-, and bifunctional linkers, coupling with protein- and non-protein macromolecules, and glycosylation. For example, mono- and bifunctional linkers are especially advantageous where contemplated polypeptides are immobilized to a solid support, or covalently coupled to a molecule that enhances immunogenicity of contemplated polypeptides (e.g., KLH, or BSA conjugation). Alternatively, contemplated polypeptides may be coupled to antibodies or antibody fragments to allow rapid retrieval of the polypeptide from a mixture of molecules. Further contemplated couplings include covalent and non-covalent coupling of contemplated polypeptides with molecules that prolong the serum half-life and/or reduce immunogenicity such as cyclodextranes and polyethylene glycols.

In a particularly contemplated aspect of the inventive subject matter, SEQ ID NO:15-SEQ ID NO:21 (the corresponding mRNA of SEQ ID NO: 1-SEQ ID NO:7) are employed in a method of detecting a neoplastic cell in a system. In one step, a predetermined quantity of an RNA comprising at least one of SEQ ID NO:15-SEQ ID NO:21 in a cell containing system is correlated with the presence of a neoplastic cell, wherein the RNA encodes an intracellular polypeptide, and in a further step, an amount of at least the predetermined quantity of the RNA is detected in the system.

In a preferred embodiment, the system is a mammal (most preferably a human) and the neoplastic cell is a prostate cancer cell in a biopsy specimen. The total RNA is extracted from the biopsy specimen, and a real time quantitative rt-PCR employing individual reactions with primer pairs specific to each of the sequences of SEQ ID NO:15-SEQ ID NO:21 is performed in parallel with a biopsy specimen known to be free of cancer cells. Biopsy specimens are determined to have a cancer cell, where the detected mRNA quantity of SEQ ID NO:15-SEQ ID NO:21 is at least 3 times higher than in the control specimen. A preferred extraction of total RNA utilizes the Quiagen BioRobot kit in conjunction with the BioRobot 9600 system, and the real time rtPCR is performed in a Perkin Elmer ABI Prism 7700.

In alternative aspects of the inventive subject matter, the method of detecting a neoplastic cell need not be limited to biopsy tissues from prostate tissue, but may employ various alternative tissues, including lymphoma tumor cells, and various solid tumor cells, so long as such tumor cells overproduce mRNA of the SEQ ID NO:15-SEQ ID NO:21. Appropriate alternative tumor cells can readily be identified by the above described method. Likewise, the system need not be restricted to a mammal, but may also include cell-, and tissue cultures grown in vitro, and tumor cells and specimens from animals other than mammals.

For example, tumor cell and tissue grown iii vitro may advantageously be utilized to investigate drug action on such cells, and the overabundance of sequences of SEQ ID NO:15-SEQ ID NO:21 may conveniently be employed as tumor marker. Alternatively, body fluids (e.g., serum, saliva, etc.) that may or may not contain tumor cells are also contemplated a suitable substrate for the method presented herein, so long as they contain to at least some extent mRNA with a sequence of SEQ ID NO:15-SEQ ID NO:21.

With respect to the detection method it is contemplated that many methods other than quantitative real time rt-PCR are also appropriate, and particularly contemplated methods include hybridization of a probe to at least one of SEQ ID NO:15-SEQ ID NO:21. It is especially contemplated that suitable probes are labeled, and depending on the physico-chemical nature of the probe, the detection process may include fluorescence detection, luminescence detection, scintigraphy, autoradiography, and formation of a dye. For example, for microscopic analysis of biopsy specimens, fluorescein modified sequence probes (complementary to at least one of SEQ ID NO:15-SEQ ID NO:21) are particularly advantageous. Fluorescence quantification may then be performed utilizing a CCD-video analysis package. Similarly, luminescence may be detected with a luminometer coupled to a microscope, or where tissue pieces are submerged in a sample cuvette, luminescence may be determined in the sample fluid. It should be appreciated that labeling of oligonucleotides and hybridization of the labeled oligonucleotide is a technique that is well known in the art, and that all known methods are generally suitable for use in conjunction with methods contemplated herein. Alternatively, the amount of mRNA may also be determined by first hybridizing a probe to the mRNA and subsequently enzymatically coupling of at least one nucleotide to the probe, and especially contemplated enzymatic additions include LCR and PCR.

In still other aspects of contemplated methods, the mRNA quantity need not necessarily be limited to at least 3 times more than in the control specimen in order to establish that the tissue has a cancer cell. For example, where the concentration of mRNA is hormone dependent, higher amounts between 3-8 fold and more may be appropriate. In contrast, where the concentration of cancer cells in the biopsy specimen is relatively low, amounts of less than 3-fold, including 1.5 to 2.9-fold and less are contemplated.

In another particularly contemplated aspect of the inventive subject matter, polypeptide of SEQ ID NO:8-SEQ ID NO:14 (encoded by SEQ ID NO: 1-SEQ ID NO:7) are employed in a method of detecting a neoplastic cell in a system. In one step, a predetermined quantity of an intracellular polypeptide comprising at least one of SEQ ID NO:8-SEQ ID NO:14 in a cell containing system is correlated with the presence of a neoplastic cell, and in a further step, an amount of at least the predetermined quantity of the RNA is then detected in the system.

In a preferred embodiment, the system is a mammal (most preferably a human) and the neoplastic cell is a prostate cancer cell or a breast cancer cell in a biopsy specimen. The biopsy specimen that is suspected to have a cancer cell is flash frozen, dissected on a microtome, and sections are mounted on microscope slides. The sections are subsequently incubated with a fluorescein labeled antibody that is directed against an epitope of at least one of the polypeptides of SEQ ID NO:8-SEQ ID NO:14. Fluorescence is detected with a fluorescence microscope coupled to a CCD-video camera and image analysis equipment. Biopsy specimens are determined to have a cancer cell, where the fluorescence signal/quantity of one or more cells is at least 3 times higher than in the control specimen.

In alternative aspects of the inventive subject matter, the method of detecting a neoplastic cell need not be limited to biopsy tissues from prostate tissue, but may employ various alternative tissues, including lymphoma tumor cells, and various solid tumor cells, so long as such tumor cells overproduce polypeptides of the SEQ ID NO:8-SEQ ID NO:14. Appropriate alternative tumor cells can readily be identified by the above described method. Likewise, the system need not be restricted to a mammal, but may also include cell, and tissue cultures grown in vitro, and tumor cells and specimens from animals other than mammals. For example, tumor cell and tissue grown in vitro may advantageously be utilized to investigate drug action on such cells, and the polypeptides of SEQ ID NO:8-SEQ ID NO:14 may conveniently be employed as a tumor marker. Alternatively, body fluids (e.g., serum, saliva, etc.) that may or may not contain tumor cells are also contemplated as suitable substrates for the method presented herein, so long as they contain to at least some extent the polypeptides of SEQ ID NO:8-SEQ ID NO:14.

With respect to detection methods, it is contemplated that many methods other than fluorescence microscopy are also appropriate, and particularly contemplated methods include specific binding of a probe to at least one of SEQ ID NO:8-SEQ ID NO:14. It is especially contemplated that suitable probes are labeled, and depending on the physico-chemical nature of the probe, the detection process may include fluorescence detection, luminescence detection, scintigraphy, autoradiography, and formation of a dye.

For example, for microscopic analysis of biopsy specimens, luciferase labeled probes are particularly advantageous in conjunction with a luminescence substrate (e.g., luciferin). Luminescence quantification may then be performed utilizing a CCD-camera and image analysis system. Similarly, radioactivity may be detected via autoradiographic or scintigraphic procedures on a tissue section, in a fluid or on a solid support. Where the probe is a natural or synthetic ligand of contemplated polypeptides, particularly contemplated ligands include molecules with a chemical modification that increase the affinity to the polypeptide and/or induce irreversible binding to the polypeptide. For example, transition state analogs or suicide inhibitors for a particular reaction catalyzed by the polypeptide are especially contemplated. Labeling of antibodies, antibody fragments, small molecules, and binding of the labeled entity is a technique that is well known in the art, and it is contemplated that all known methods are generally suitable for use in conjunction with methods contemplated herein. Furthermore, the probe need not be limited to a fluorescein labeled antibody, and alternative probes include antibody fragments (e.g., Fab, Fab′, scFab, etc.).

In still other aspects of contemplated methods, the polypeptide quantity need not necessarily be limited to at least 3 times more than the control specimen in order to establish that the tissue has a cancer cell (e.g., where the control reads 100 ng, three times more than the control means 300 ng). For example, where the concentration of the polypeptide is hormone dependent, higher amounts between 3-8 fold and more may be appropriate. In contrast, where the concentration of cancer cells in the biopsy specimen is relatively low, amounts of less than 3-fold, including 1.5 to 2.9-fold and less are contemplated.

It should further be appreciated that the polynucleotides of SEQ ID NO:1-SEQ ID NO:7 and SEQ ID NO:15-SEQ ID NO:21 may be employed as a therapeutic modality in an antisense DNA/RNA based therapy. Anti-sense therapy, for example, could be employed to inhibit, up-, or down-regulate transcription or translation of the genes corresponding to SEQ ID NO:1-SEQ ID NO:7. It should further be appreciated that an anti-sense approach may also include regulatory sequences associated with SEQ ID NO:1-SEQ ID NO:7 such as transcription enhancers, hormone responsive elements, ribosomal- and RNA polymerase binding sites, etc., which may be located upstream or downstream of SEQ ID NO:1-SEQ ID NO:7, and may have a distance of several ten base pairs to several ten thousand base pairs.

Alternatively, the polypeptides of SEQ ID NO:8-SEQ ID NO:14 may also be employed in an antibody based therapy or a small molecule drug therapy directed towards the polypeptides of SEQ ID NO:8-SEQ ID NO:14. For example, antibody based therapy could be employed to neutralize, or remove corresponding polypeptides of SEQ ID NO:8-SEQ ID NO:14 in-vivo, or to interfere with one or more cellular functions of contemplated polypeptides.

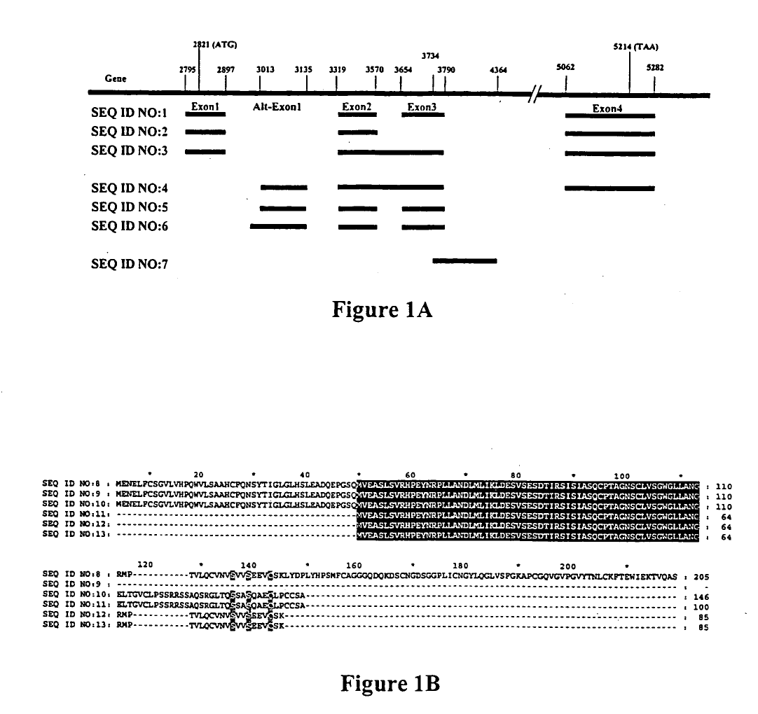

FIGS. 1A and 1B show a schematic and an amino acid based alignment between the cDNAs of SEQ ID NO:1-SEQ ID NO:7, in which SEQ ID NO:1 is the full-length cDNA and SEQ ID NO:2-SEQ ID NO:7 are splicing variants of SEQ ID NO: 1. In the amino acid based alignment SEQ ID NO:8 is the corresponding polypeptide to SEQ ID NO:1, and SEQ ID NO:9-SEQ ID NO:14 are the corresponding polypeptides to SEQ ID NO:2-SEQ ID NO:6.

### EXAMPLES

The following examples illustrate the isolation and cloning of the sequences of SEQ ID NO:1-SEQ ID NO:7 form normal prostate tissue and from prostate cancer cells. SEQ ID NO:8-SEQ ID NO:14, and SEQ ID NO:15-SEQ ID NO:21 are computer generated transcriptions and translations of SEQ ID NO:1-SEQ ID NO:7, respectively.

The following examples also illustrate a general method of identifying differentially expressed genes in a target tissue, in which in one step a target tissue-specific cDNA library is provided that has a plurality of tissue-specific genes obtained by suppression subtractive hybridization. In a subsequent step, a predetermined quantity of tissue-specific genes is immobilized on a solid phase to form a tissue-specific cDNA array, and a first nucleic acid preparation is hybridized to a first tissue-specific cDNA array to create a first hybridization pattern, wherein the first preparation is prepared from the target tissue without previously exposing the target tissue to a compound. In a further step, a second nucleic acid preparation is hybridized to a second tissue-specific cDNA array to create a second hybridization pattern, wherein the second preparation is prepared from the target tissue after previously exposing the target tissue to a compound. In yet a further step, the first and the second hybridization pattern are then compared to identify differentially expressed genes. This general method is especially contemplated where the compound comprises a hormone, or various other suitable ligands.

Suppression Subtraction of Prostate Specific Genes

cDNA derived from poly(A)+ RNA of 10 different normal human tissues were subtracted against normal human prostate cDNA using suppression subtraction hybridization (SSH) (Diatchenko, L., Lau, Y.-F-C., Campbell, A. P., Chenchik, A., Moqadam, F., Huang, B., Lukyanov, S., Lukyanov, K., Gurskaya, N., Sverdlov, E. D., Siebert, P. D. (1996). Proc. Natl. Acad. Sci. USA 93, 6025-6030), and the resulting cDNA fragments were cloned into an appropriate vector. SSH was performed as described (Clontech PCR-Select Cloning Kit) using prostate poly (A)+ RNA against a pool of poly(A)+ RNA obtained from ten normal human tissues (heart, brain, placenta, lung, liver, skeletal muscle, kidney, spleen, thymus, and ovary). Upon secondary PCR amplification (12 cycles), reactions were extracted with phenol/chloroform and DNA was precipitated with EtOH.

The pellet was washed once with 70% EtOH. After drying, the DNA pellet was dissolved in 0.2×TE or dH₂O and cut with Rsa1 in a 20 ul reaction for 2 hrs at 37C to excise adaptors. After digestion, reactions were run on a 1.5% agarose gel, with molecular size markers on one side, at 5 V/cm, 40 min. The adopter bands are excised and discarded, and cDNA bands were cut out and purified (QAIEX gel DNA purification kit) after running the gel backwards to concentrate the cDNA.

The purified DNA was subcloned into EcoRV-cut, dephosphorylated pZERO vector from Invitrogen. DH 10B electrocompetent cells (>10¹⁰ efficiency) were transformed with a ⅕ dilution of 1 μl of the ligation mix.

Colonies were picked and the presence of cDNA inserts confirmed by PCR with T7 and SP6 primers directly from the colonies. 10% of reactions were run on a 1.5% agarose gel to visualize amplified products. The colonies with inserts were grown and glycerol stocks (15%) were prepared and stored at −80C.

Reverse Northern Blot and Sequence Analyses

Clones from the SSH library were amplified by PCR and spotted on nylon filters in 96-well format to generate two identical blots for each set of 92 clones (the remaining four spots were used for positive and negative controls). For probe preparation, the androgen-responsive prostate cancer cell line LNCaP (Horoszewicz, J. S., Leong, S. S., Kawinski, E., Karr, J. P., Rosenthal, EL, Chu, T. M., Mirand, E. A., and Murphy, G. P. (1983). Cancer Res. 43, 1809-1818) was employed that was either untreated [the (−) probe] or treated with the synthetic androgen R1881 for 24 hours [the (+) probe]. Poly(A)+ RNA was isolated from these cells and was used to make the ³²P-labeled probes. After hybridization with the (−) and (+) probes, clones showing differential hybridization were selected for further analysis (i.e., confirmation by a secondary reverse northern blot, and northern blotting).

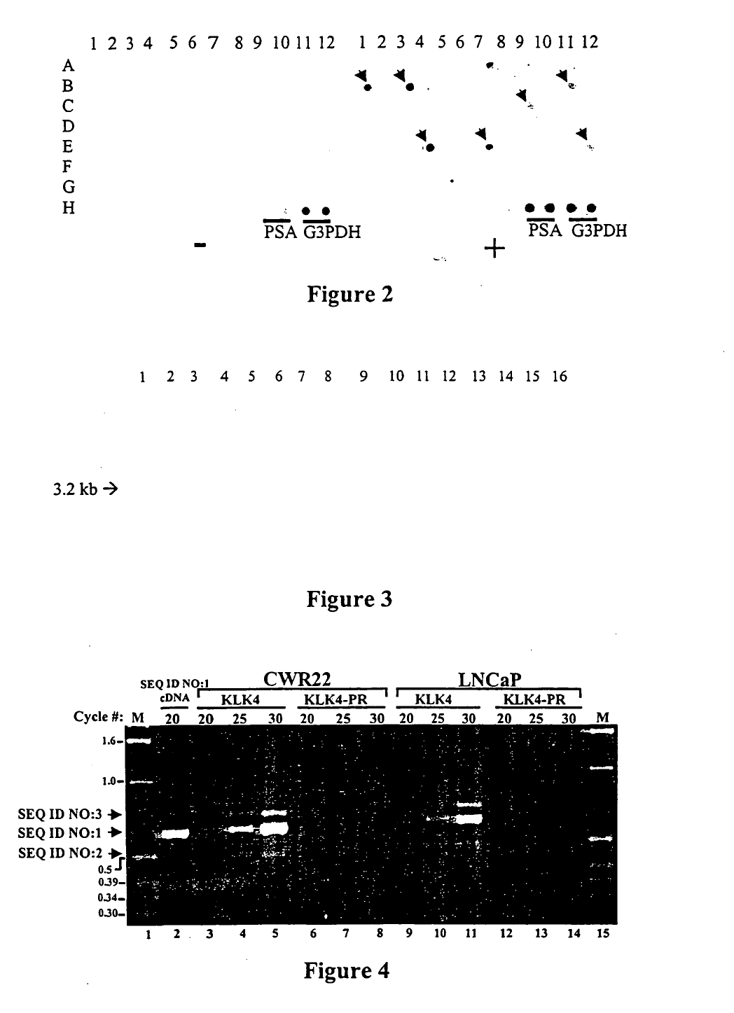

Reverse northern screening on the cDNA clones were done essentially as described elsewhere (Hedrick, S. M., Cohen, D. I., Neilson, E. A., Davis, M. M. (1984) Nature 308, 149-153; Sakaguchi N, Berger CN, Melchers F (1986). EMBO J5. 2139-2147). DNA (approximately 400 ng) from PCR amplification in step 6 was diluted in 200 μl of 0.4M NaOH, 10 mM EDTA and mixed well by pipetting. After incubation at 95° C. for 5-10 min, the tubes were chilled on ice. Denatured DNA was blotted on two separate pieces of Zeta Probe GT+ membrane (Bio-Rad) using a dot-blot apparatus (Bio-Rad). Positive [Prostate specific antigen (PSA) cDNA) and negative [glyceraldehydes 3-phosphate dehydrogenase (G3PDH) cDNA) controls are included on each blot in duplicate. Membranes were rinsed with 2×SSC, air dried, and then baked at 80° C. for 30 min. A typical example of a reverse northern analysis is shown in FIG. 2. In each blot pair, PSA and G3PDH are included as positive and negative controls, respectively. It should be noted that there was substantial increase in PSA hybridization in the (+) blot (probe prepared from cells that have been stimulated by androgens) compared with the (−) blot (probe prepared from untreated cells), whereas there was no significant change in hybridization of G3PDH between the two blots. Arrowheads indicate differentially expressed clones.

As a control to verify the prostate specific nature of isolated sequences, positive clones were tested in a standard northern blot against RNA preparations of multiple non-prostate tissues and a typical blot is shown in FIG. 3. Lanes 1-10, and 12-16 are RNA preparations from non-prostate tissues, lane 11 is a RNA preparation from prostate, lane 12 is a RNA preparation from testis.

Probes: The probes were random-prime radiolabeled using standard laboratory procedures. Unincorporated nucleotides were removed using prespun G25 columns (Bio-Rad), and specific activity was typically over 5×10⁸ cpm/μg.

Hybridization: 25 ml Hybridization mix (7% SDS, 0.5 M NaHPO₄, 1 mM EDTA) at 65° C. is prewarmed, and 12.5 ml were used for prehybridization of each membrane for 5-10 min at 65° C. Probes were heat denatured at 95° C. for 3-5 min and transferred to the prehybridization mix at 65° C. Hybridization was done at 65° C. overnight.

Washing: Wash solution I (2×SSC and 1% SDS) and wash solution 11 (0.1×SSC and 0.5% SDS) were prewarmed. Membranes were washed once with Solution I and then Solution II for 30 min at 65° C., covered with plastic wrap and exposed to phoshorimager screen.

Selection: Clones showing differential expression between the (−) and (+) blots were picked. A secondary round of reverse northern analysis is performed for confirmation by spotting each clone in duplicate on each blot. To confirm hormone dependence, a time course of R1881 induction of LNCaP cells as well as the CWR22 xenograft model upon androgen ablation (Wainstein, M. A., He, F., Robinson, D., Kung, H. J., Schwartz, S., Giaconia, J. M., Edgehouse, N. L., Pretlow, T. P., Bodner, D. R., Kursh, E. D., and Pretlow, T. G. (1994). Cancer Res. 54, 6049-6052) and the androgen-independent CWR22R relapsed xenograft (Nagabhushan, M., Miller, C. M., Pretlow, 1. P., Giaconia, J. M., Edgehouse, N. L., Schwartz, S., Kung, H. J., deVere White, R. W., Gumerlock, P. H., Resnick, M. I., Amini, S. B., and Pretlow, T. G. (1996). Cancer Res. 56, 3042-6) was used.

Sequence analysis: Sequence analysis was performed by the dideoxy chain termination methods using an ABI automated sequencer. It should be appreciated that many more androgen responsive, differentially expressed genes can be identified and isolated using the cloning strategy outlined above, including genes expressed during various growth and developmental phases of a diseased prostate, and genes expressed as a result of a drug regimen. Moreover, it is contemplated that not only differentially expressed prostate cancer genes can be identified and isolated, but also genes involved in other diseased states of human prostate, including benign prostate hyperplasia, etc.

Isolation of Splice Variants (SEQ ID NO: 2-SEQ ID NO: 7)

Poly(A)+ RNA was isolated from LNCaP cells treated with R1881 (a synthetic androgen) and from androgen dependent prostate cancer xenograft CWR22 grown in nude mice in the presence of androgens. cDNA was prepared and subjected to PCR using SEQ ID NO:1 specific primers and a primer pair designed to amplify the previously published prostase. The respective 5′-primers were located around the translation start site, while the 3′-primer for all reactions was located around the stop codon. Reaction products were loaded onto an agarose gel and separated as shown in FIG. 4. Lane 1 is a marker, lane 2 is positive control with SEQ ID NO:1 as template. Lanes 3-5 are PCR products from CWR.22 cells with SEQ ID NO:1 specific primers, while lanes 6-8 are PCR products from CWR22 cells with prostase specific 5′-primer. Lanes 9-11 are PCR products from LNCaP cells with SEQ ID NO:1 specific primers, and lanes 12-14 are PCR products from LNCaP cells with prostase specific primers. Lane 15 is marker.

Only reactions with SEQ ID NO: 1 specific primers yielded detectable PCR products, with a major band at 680 bp (SEQ ID NO:1), and two additional bands at about 500 bp (SEQ ID NO:2) and 750 bp (SEQ ID NO:3). When primers for 5′-RACE analysis were employed, four additional PCR products were obtained, corresponding to SEQ ID NO:4, SEQ ID NO:5, and SEQ ID NO:6, respectively. All bands were sequenced to confirm their identity. SEQ ID NO:7 was obtained both as a 3′-RACE product as well as a distinct clone from the PSL library.

Localization of Intracellular Proteins Encoded by SEQ ID NO: 1-SEQ ID NO:3

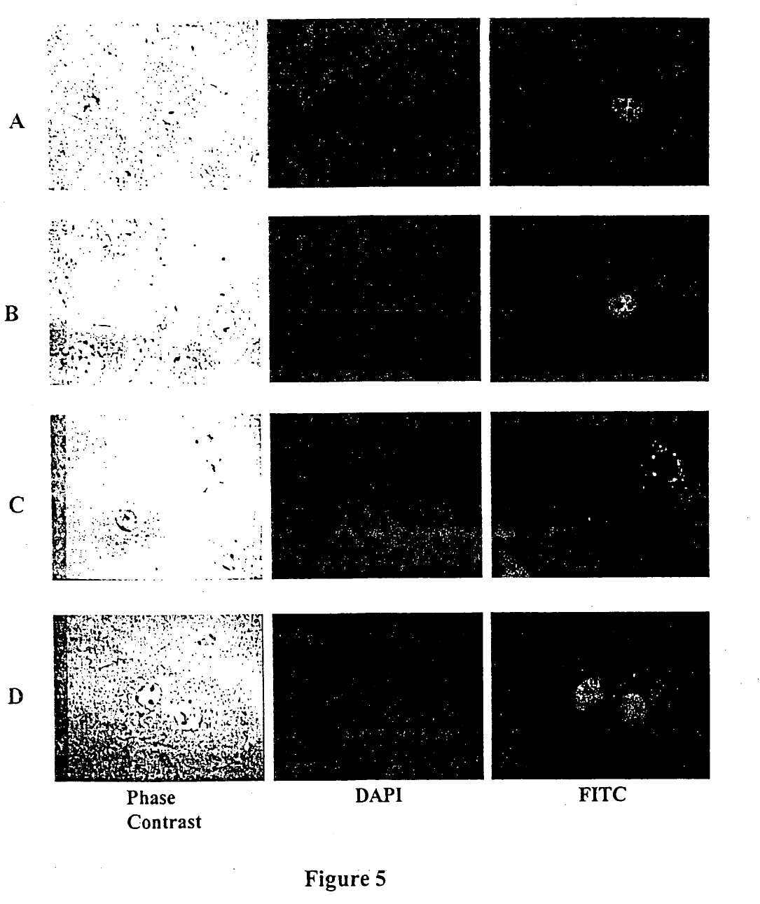

To gain insight into the subcellular location of the polypeptides SEQ ID NO:8-SEQ ID NO:10 (encoded by SEQ ID NO:1-SEQ ID NO:3), C-terminal fusion constructs with GFP were produced in COS7 cells. The cells were fixed, stained with DAPI and visualized by phase contrast or fluorescence microscopy and representative images are shown in FIG. 5. Photographs in lane A depict a GFP fusion protein with SEQ ID NO:9, the photographs in lane B depict a GFP fusion protein with SEQ ID NO:10, the photographs in lane C depict a GFP fusion protein with SEQ ID NO:8, and the photographs in lane D depict a GFP protein as a control. The polypeptide of SEQ ID NO: 8 displayed strong granular fluorescence predominantly around the nucleus, while the polypeptide of SEQ ID NO:9 and SEQ ID NO:10 showed exclusively nuclear and predominantly nuclear localization, respectively. Due to the lack of an identifiable leader sequence that would indicate an export of the polypeptides of SEQ ID NO:11-SEQ ID NO:14, it is contemplated that the sequences SEQ ID NO:11-SEQ ID NO:14 are also intracellular proteins.

Regulation of Expression by multiple Hormones

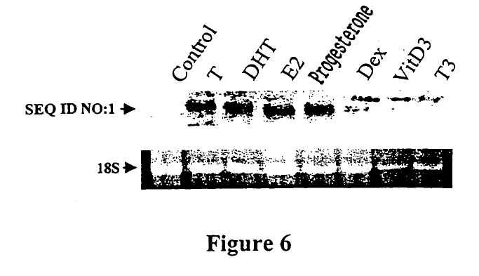

Untreated LNCaP cells and hormone treated LNCaP cells were employed to determine the hormone dependence of expression of SEQ ID NO:1. Treatment was as follows: Testosterone (T) at 10⁻⁸M, dihydrotestosterone (DHT) at 10⁻⁸M, estradiol (E2) at 10⁻⁸M, progesterone (P) at 10⁻⁸M, dexamethasone (Dex) at 10⁻⁷M, 1,25-dihydroxy-vitamin D3 (VitD3) at 10⁻⁸M, and triiodothyronine (T3) at 10⁻⁷M. The total RNA of the treated cells was isolated and used in a northern blot analysis with radiolabeled SEQ ID NO:1 as probe. FIG. 6 shows the results of an autoradiograph of a northern blot as described above. 18S-RNA is shown as control for RNA integrity and loading. The relative induction of SEQ ID NO:1 is indicated at the bottom of the lanes as determined by phosphorimager analysis. It is contemplated that SEQ ID NO:2-SEQ ID NO:7 are splice variants of SEQ ID NO:1, and consequently it is contemplated that the expression of all of SEQ ID NO:1-SEQ ID NO:6 is hormone dependent, and particularly contemplated hormones include androgens, progesterones, estrogens and glucocorticoids.

Thus, specific embodiments and applications of methods and applications of differentially expressed genes in prostate cancer cells have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A method of detecting a neoplastic cell in a sample comprising determining the amount of a polypeptide comprising the sequence of any one of SEQ ID NOs: 10, 11 and 14 in said sample relative to a non-neoplastic control, wherein an increase in the amount of said polypeptide in said sample relative to the amount of said polypeptide in said control identifies said sample as having at least one neoplastic cell.

2. The method of claim 1, wherein said increase is at approximately 3 fold.

3. The method of claim 1, wherein said increase is between 3 to 8 fold.

4. The method of claim 1, wherein said increase is between 1.5 and 2.9 fold.

5. The method of claim 1, wherein said sample is from breast or prostate tissue.

6. The method of claim 1, wherein said sample comprises at least one breast or prostate cell.

7. The method of claim 1, wherein said sample is taken from a mammal.

8. The method of claim 7, wherein said mammal is a human.

9. The method of claim 1, wherein said sample is a biopsy specimen, an in vitro cell culture, an in vitro tissue culture, or body fluid.

10. The method of claim 1, wherein said determining comprises specifically binding a probe to said polypeptide.

11. The method of claim 10, wherein said probe is selected from the group consisting of an antibody, an antibody fragment, a natural ligand of the polypeptide, and a synthetic ligand of the polypeptide.

12. The method of claim 10, wherein said probe is detectably labeled.

13. The method of claim 10, wherein said probe is detected by a process selected from the group consisting of fluorescence detection, luminescence detection, scintigraphy, autoradiography, and formation of a dye.

14. The method of claim 1, wherein said polypeptide comprises the sequence of SEQ ID NO:10.
